(19) United States
(12) Patent Application Publication
(10) Pub. No.: US 2003/0118591 A1
(43) Pub. Date: Jun. 26, 2003

(54) Passive hyperimmune antibody therapy in the treatment of anthrax

(76) Inventor: Joshua Levy, North Hollywood, CA (US)

Correspondence Address: FULWIDER PATTON LEE & UTECHT, LLP, HOWARD HUGHES CENTER, 6060 CENTER DRIVE, TENTH FLOOR, LOS ANGELES, CA, 90045, US

(21) Appl. No.: 10/027,478

(22) Filed: Dec. 21, 2001

(51) Int. Cl. A61K 39/40; A61K 39/07

(52) U.S. Cl. 424/165.1; 424/246.1

(57) ABSTRACT

A method of treatment of severe anthrax infection particularly inhalation pneumonia or gastrointestinal anthrax antigen by the passive transfer to infected patients of plasma or plasma fractionated derivatives, such as gammaglobulins or antibodies, monclonal or polyclonal, with high titer neutralizing antibodies against Bacillus anthracis or its toxins. The plasma or fractionated plasma derivatives are derived from previously vaccinated individuals with anthrax vaccine, or any antigen or toxin antigen of Bacillus anthracis, including protective antigen (PA), lethal factor (LF) and/or oedema factor (OF).

### BACKGROUND OF THE INVENTION

The present invention relates generally to the treatment of severe anthrax with the passive transfer to infected patients of human plasma or plasma fractionated derivatives such as gammaglobulins or antibodies, with neutralizing antibodies against Bacillus anthracis or its toxins. Polyclonal antibodies are derived from plasma collected from individuals vaccinated with anthrax vaccine or antigens from the anthrax bacillus or any of the components or antigens of the toxins produced by the anthrax bacillus.

Anthrax poses a significant threat to the human population throughout the world as an agent of biological warfare and terrorism. Anthrax occurs globally in temperate zones, but is more often a risk in developing countries which have less standardized public health programs in place. Humans can become infected with anthrax through the handling of products or consumption of undercooked meat from infected animals such as cattle, sheep and goats. Infection can also result from inhalation of bacterial spores originating from contaminated animal products (i.e., wool) or through the intentional release of bacterial spores during a bioterrorist attack.

Anthrax infection in humans may assume one of the following three forms: (1) cutaneous; (2) inhalation; and (3) gastrointestinal anthrax. Cutaneous anthrax accounts for approximately 95% of anthrax infections which occur when the bacterial spores enter a cut or abrasion directly on the skin, such as during the handling of various contaminated products of infected animals. Those exposed to this form of anthrax can be treated with antibiotics, such as penicillin, ciprofloxacin, and doxycycline. However, in humans, early antibiotic treatment of cutaneous anthrax is essential as any delay decreases one's chances for survival. Anthrax in its inhalational form is the most severe of the three and many if not most are fatal. Early symptoms of inhalational anthrax are similar to a common cold, but after several days the infected person suffers severe breathing problems and shock. The third form of infection, gastrointestinal anthrax, occurs from the consumption of contaminated meat and is typically characterized by an acute inflammation of the intestinal tract. Nearly 25% to 60% of gastrointestinal anthrax cases are fatal. As set forth above, early treatment of anthrax is necessary for a successful recovery, as otherwise, if left untreated, anthrax is highly fatal.

A vaccine for anthrax, manufactured and distributed by BioPort Corporation of Lansing, Mich., is presently licensed for use in humans and is reported to be about 93% effective in protecting against cutaneous anthrax. The anthrax vaccine consists of a cell-free filtrate, containing protective antigen and alum, free of any dead or live bacteria in the preparation. The advisory committee on immunization practices of the federal Center for Disease Control and Prevention (CDCP) recommends vaccination to only a select group of individuals, such as military personnel, who stand a high risk of being exposed to the bacterium (as a biological warfare weapon) when deployed to certain areas throughout the world.

The main pathogenic factors of Bacillus anthracis consist of a poly-D-glutamic acid capsule and anthrax toxin. The anthrax toxin includes three distinct proteins, acting in concert—two enzymes, lethal factor (LF) and oedema factor (OF) (an adenylate cyclase), and a protective antigen (PA). A patient infected with the bacterium Bacillus anthracis, gives rise to anthrax and results in the secretion of the tripartite toxin (anthrax toxin), the causative agent of anthrax, which helps the bacterium evade the immune system and kill the host during systematic infection. An antigen is any substance which generates an immune response leading to acquired immunity when introduced into a host animal or human. In particular, an antigen may be either a soluble substance, such as a bacterial toxin or serum protein, or it may be particulate in nature, such as a bacterial cell. Generally, the greater degree to which an antigen is foreign to the person being immunized, in terms of its chemical composition and structure, the greater its effectiveness in triggering an immune response. Located on the surface (and in some cases the interior) of the substance acting as an antigen, such as a bacterial or body cell, or a virus, are a number of reactive sites which impart specificity to the immune response by reacting with an antibody or a lymphocyte.

The formation of specific antibodies circulating in the bloodstream, or an increase in the number of specifically reactive cells (lymphocytes), or both, occurs as a result of the immune response. The specific antibodies and specifically reactive lymphocytes both react to the antigen that functions as the immunizing agent. By acquiring immunity in this manner, the body is able to destroy or immunize invading pathogens. Thus, a person's acquired immunity acts as the predominant line of internal defense against such invading pathogens.

The passive transfer of antibodies in the form of whole plasma or fractionated preparations, such as gammaglobulins, has been used for both the prophylaxis and treatment of infection in patients with primary immunodeficiency, as well as infections associated with transplantation, chronic leukemia, premature birth, and surgery (See Berkman, S A, et al., Ann. Intern. Med. 112:278, 1990; Ziegler, E J, et al., New England J. of Med. 307;1225, 1982; Gordon, D S, Am. J. Med. 83:1m 1987, [suppl. 4A]; Winston, D J, et al., Ann. Intern. Med. 106:12, 1987; The National Institute of Child Health and Human Development Intravenous Immunoglobulin Study Group, New England J. of Med. 325:73, 1991). Hyperimmune antibodies directed against a single organism passively transfered to recipients has been particularly useful in the treatment of cytomegalovirus, hepatitis B, tetanus, vaccinia, and herpes (Orenstein, W A, et al., J. Pedicat. 98:368, 1981; Snydman, D R, et al., New England J. of Med. 317:1049, 1987; Beasley, et al., Lancet 2:388, 1981).

In animal studies in guinea pigs infected with lethal doses of virulent anthrax spores, the passive transfer of hyperimmune serum from animals vaccinated with anthrax protective antigen vaccine conferred protection from a fatal outcome. The titer of neutralizing antibody to protective antigen in the passively transferred sera correlated with the degree of protection from death (Reuveny, S, et al., Infect. Immune 69(5):2888-93, 2001).

Another study in guinea pigs evaluated the protective effects of passive transfer of polyclonal antisera derived from animals vaccinated with anthrax protective antigen in infected recipients with Bacillus anthracis spores as compared to infusion of monoclonal antibodies with activity against PA, LF or OF. The study found that only polyclonal antibodies from PA vaccinated animals gave significant protection from death in the infected animals and that the monclonal antibodies failed to protect the infected animals (Little, et al., Infect. Immune 65(12):5171-5, 1997).

Over 500,000 military personnel have been vaccinated with anthrax (PA), and many have generated high titers of polyclonal neutralizing antibody to anthrax (PA) in their plasma. Plasma collected from these individuals or fractionated gammaglobulins from their plasma could offer significant therapeutic benefit to serious anthrax infected human individuals, particularly when antibodies are given in combination with appropriate antibiotics.

Passive transfer of hyperimmune antibodies with neutralizing antibody titers against anthrax toxin antigens would complement antibiotic treatment, as it would be able to neutralize circulating toxin—often the cause of death in infected individuals. Antibiotics may kill the anthrax bacillus, but they cannot neutralize already circulating anthrax toxin. Often when symptoms of pneumonic anthrax infection first appear, a lethal dose of anthrax toxin is already circulating and antibiotics alone may not be sufficient to save the life of the individual. Thus, it is desirable to provide a method for treating individuals infected with the anthrax bacterium using passive hyperimmune antibody therapy in combination with antibiotics for the most effective treatment possible. The present invention meets these needs and others.

### SUMMARY OF THE INVENTION

Briefly, and in general terms, the present invention provides a method of treatment of severe anthrax infection by the passive transfer to infected patients of plasma or plasma fractionated derivatives, such as gammaglobulins or antibodies, monoclonal or polyclonal, with neutralizing antibodies against Bacillus anthracis or its toxins.

The principal objective of this invention is the protection from a fatal outcome in patients with life-threatening anthrax infection by passively transferring to infected patients high titer neutralizing antibodies to anthrax toxin. The plasma or fractionated plasma derivatives, such as gammaglobulins, are derived from individuals previously vaccinated with anthrax vaccine or any of the Bacillus antigens or toxin antigens including protective antigen (PA), lethal factor (LF) or oedema factor (OF).

Plasma is collected by manual or automated plasmapheresis from anthrax vaccinated donors with high neutralizing titers of antibodies to the Bacillus or its toxins who meet FDA criteria as normal donors and who test negative for all infection markers, i.e., Hepatitis B and C, and HIV. Donors may donate up to 800 cc of plasma twice a week. Plasma from at least fifty donors are pooled into large batches and either sterilized by solvent detergent (Prince, A M, et al., Cancer Res. [Supp] 45:4592S, 1985) and used as a therapeutic plasma infusion, or fractionated by any of the common methods of Cohn fractionation or chromatography fractionation followed by sterilization by solvent detergent to produce pure gammaglobulin which can be given therapeutically as an IV infusion or intramuscular injection to severely infected patients.

These and other aspects and advantages of the invention will become apparent from the following detailed description, which illustrates by way of example the features of the invention.

### DETAILED DESCRIPTION OF THE PREFERRED EMBODIMENTS

The present invention is directed to a method of treatment for severe anthrax infection by the passive transfer to infected patients of plasma or plasma fractionated derivatives, such as gammaglobulins or antibodies, monoclonal or ployclonal, possessing a high titer of neutralizing antibodies to Bacillus anthracis or any of its toxins.

In a preferred embodiment, the plasma source consists of plasma derived from individuals previously vaccinated with anthrax vaccine or any antigens from Bacillus anthracis, including any toxin antigens—protective antigen (PA), lethal factor (LF) or oedema factor (OF). Likely candidates supplying the plasma source include the over 500,000 U.S. military personnel to date who have been vaccinated with anthrax PA and hence have generated high titers of polyclonal neutralizing antibody to anthrax PA in their plasma. As noted earlier, military personnel are one of the select groups recommended by the federal Center for Disease Control and Prevention (CDCP) for anthrax vaccination as such individuals are highly susceptible to being exposed to the bacterium (i.e., as a biological warfare weapon) when deployed to particular countries while on active duty. Prospective donors of the plasma source for the present invention must satisfy FDA criteria as normal donors who test negative for all designated infection markers, such as Hepatitis B and C, and HIV. Qualified donors may donate weekly up to 800 cc of plasma. The collection of plasma from qualified donors occurs by manual or automated plasmapheresis which is known in the art.

Preferably, plasma from a minimum of fifty donors is pooled into large batches and can be sterilized by the solvent detergent method in accordance with Prince, A M, et al., Cancer Res. [Supp.] 45:45925, 1985, and incorporated herein by reference. Following sterilization by the solvent detergent method, the processed plasma can be administered as a therapeutic plasma infusion into the infected patient.

In another preferred embodiment, the pooled plasma source can be fractionated to produce pure gammoglobulin by the methods of Cohn fractionation or chromatography fractionation which are known in the art. The fractionated antibodies following sterilization by solvent detergents can be administered as a therapeutic IV infusion or intramuscular injection to severely infected patients.

It will be apparent from the foregoing that while particular forms of the invention have been illustrated and described, various modifications can be made without departing from the spirit and scope of the invention. Accordingly, it is not intended that the invention be limited, except as by the appended claims.

What is claimed is:

1. A method of treatment for patients severely infected with anthrax using passive hyperimmune antibody therapy, comprising:
deriving a supply of plasma from previously vaccinated individuals;
processing the plasma derived therefrom to provide a preparation of gammaglobulins having a high titer of neutralizing antibodies to anthrax, the processed plasma being sterilized and free of any impurities; and
administering the processed plasma to the infected patient.

2. The method of claim 1, wherein anthrax is an acute infectious disease of the spore-forming bacterium Bacillus anthracis.

3. The method of claim 2, wherein the plasma is derived from individuals previously vaccinated with anthrax vaccine.

4. The method of claim 2, wherein the plasma is derived from individuals previously vaccinated with an antigen of Bacillus anthracis.

5. The method of claim 2, wherein the plasma is derived from individuals previously vaccinated with at least one of any component and any toxin produced by Bacillus anthracis.

6. The method of claim 5, wherein the toxin antigens are selected from the group consisting of protective antigen, lethal factor, and oedema factor.

7. The method of claim 1, wherein the plasma derived from vaccinated individuals is collected by at least one of the methods of manual and automated plasmapheresis.

8. The method of claim 1, wherein the processed plasma is derived from at least fifty individuals and is pooled into large batches prior to administering the processed plasma to the infected patient.

9. The method of claim 1, wherein processed plasma is fractionated to produce gammaglobulin.

10. The method of claim 9, wherein the processed plasma is fractionated by Cohn fractionation.

11. The method of claim 9, wherein the processed plasma is fractionated by chromatography fractionation.

12. The method of claim 9, wherein the fractionated plasma is administered therapeutically by IV infusion in a safe and effective dose.

13. The method of claim 9, wherein the fractionated plasma is administered therapeutically by intramuscular injection in a safe and effective dose.

14. The method of claim 1, wherein the processed plasma is administered therapeutically by plasma infusion in a safe and effective dose.
